(19) United States
(12) Patent Application Publication
(10) Pub. No.: US 2023/0380465 A1
(43) Pub. Date: Nov. 30, 2023

(54) METHOD FOR PROCESSING VANILLA POD

(71) Applicant: Sung-Yen Liu, Taitung City (TW)

(72) Inventor: Sung-Yen Liu, Taitung City (TW)

(21) Appl. No.: 17/940,385

(22) Filed: Sep. 8, 2022

(30) Foreign Application Priority Data
May 24, 2022 (TW) 111119326

(51) Int. Cl. A23L 29/00; A61L 2/10; A61L 2/00

(52) U.S. Cl. CPC A23L 29/06; A61L 2/10; A61L 2/0023

(57) ABSTRACT

A method for processing a vanilla pod includes placing the vanilla pod in a container, followed by sealing to keep away from air, subjecting the vanilla pod in the sealed container to a killing treatment, so as to destroy cellular tissues of the vanilla pod and release enzymes from the cellular tissues, and subjecting the killed vanilla pod in the sealed container to a heating treatment at a temperature ranging from 40° C. to 60° C., so that the enzymes react with components in the killed vanilla pod to synthesize aroma molecules.

### CROSS-REFERENCE TO RELATED APPLICATION

This application claims priority of Taiwanese Invention Patent Application No. 111119326, filed on May 24, 2022.

### FIELD

The present disclosure relates to a method for processing a vanilla pod.

### BACKGROUND

Vanilla is an expensive spice that is often used in desserts, drinks, and main course of a meal. The unique aroma of vanilla can optimize the overall presentation oi the meal, thereby achieving the finishing touch. Vanilla is expensive because its processing steps are time-consuming and cumbersome.

Conventional methods for processing vanilla include the steps of picking native vanilla, killing, sweating, drying, and conditioning. These processing steps take several months, and if there is negligence in temperature, humidity, and ambient air conditions during the processing, the expected aromatic molecules would not be produced, and might even cause problems such as spoilage, formation of molds, etc. In spite of the aforesaid, there is still a need to develop a method for processing a vanilla pod, which can reduce labor costs and product wastes.

### SUMMARY

Therefore, in a first aspect, the present disclosure provides a method for processing a vanilla pod, which can alleviate at least one of the drawbacks of the prior art. The method includes:
(a) placing the vanilla pod in a container, followed by sealing to keep away from air;
(b) subjecting the vanilla pod in the sealed container to a killing treatment, so as to destroy cellular tissues of the vanilla pod and release enzymes from the cellular tissues; and
(c) subjecting the killed vanilla pod in the sealed container to a heating treatment at a temperature ranging from 40° C. to 60° C., so that the enzymes react with components in the killed vanilla pod to synthesize aroma molecules.

In a second aspect, the present disclosure provides a method for processing a vanilla pod, which can alleviate at least one of the drawbacks of the prior art. The method includes:
(a) subjecting the vanilla pod to a killing treatment, so as to destroy cellular tissues of the vanilla pod and release enzymes from the cellular tissues;
(b) placing the killed vanilla pod obtained in step (a) in a container, followed by sealing to keep away from air; and
(c) subjecting the killed vanilla pod in the sealed container to a heating treatment at a temperature ranging from 40° C. to 60° C., so that the enzymes react with components in the killed vanilla pod to synthesize aroma molecules.

### BRIEF DESCRIPTION OF THE DRAWINGS

The above and other objects, features and advantages of the present disclosure will become apparent with reference to the following detailed description and the exemplary embodiments taken in conjunction with the accompanying drawings, in which:

FIG. 1 is a flow chart illustrating a first embodiment of a method for processing a vanilla pod according to the present disclosure;

FIG. 2 is a schematic view illustrating steps (a) to (d) of the first embodiment;

FIG. 3 is a schematic view illustrating a variation of the first embodiment;

FIG. 4 is a flow chart illustrating a second embodiment of a method for processing a vanilla pod according to the present disclosure; and

FIG. 5 is a schematic view illustrating steps (a) to (d) of the second embodiment.

### DETAILED DESCRIPTION

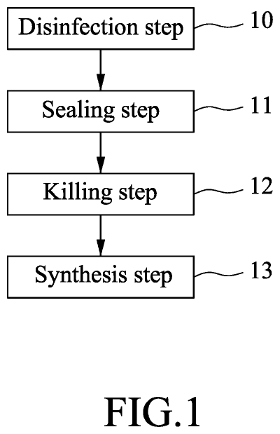

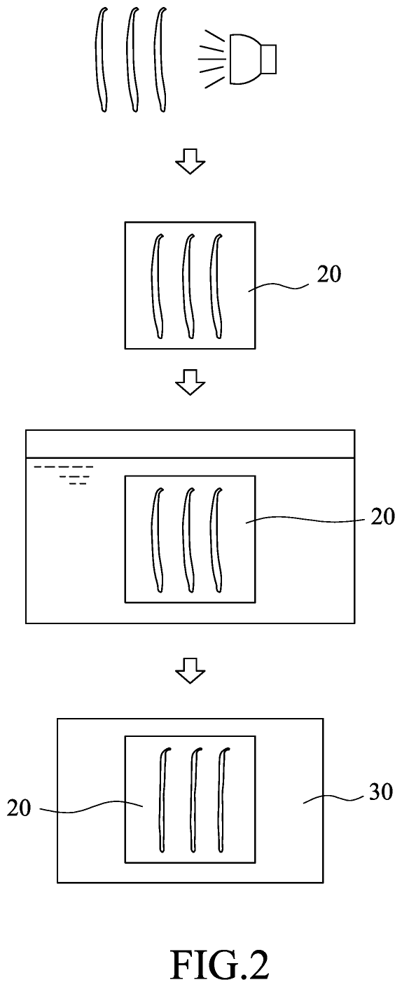

Referring to FIGS. 1 and 2, a first embodiment of a method for processing a vanilla pod according to the present disclosure includes:
(a) subjecting the vanilla pod to a disinfection treatment, so as to eliminate bacteria and mold on a surface of the vanilla pod;
(b) placing the disinfected vanilla pod in a container, followed by sealing to keep away from air;
(c) subjecting the disinfected vanilla pod in the sealed container to a killing treatment, so as to destroy cellular tissues of the vanilla pod and release enzymes from the cellular tissues; and
(d) subjecting the killed vanilla pod in the sealed container to a heating treatment at a temperature ranging from 40° C. to 60° C., so that the enzymes react with components in the killed vanilla pod to synthesize aroma molecules.

The details of the steps are described below.

In this embodiment, 10 freshly harvested vanilla pods are used for processing the vanilla pod. Naturally grown vanilla pods will have different 2D sizes, and the difference in size does not affect the implementation of the method of the present disclosure.

In step (a) of this embodiment. (which is referred to as “the disinfection step 10” hereinafter), the disinfection treatment may be conducted using a disinfection method selected from the consisting of ultraviolet (UV) light disinfection, alcohol disinfection, hypochlorous acid disinfection, and combinations thereof. In certain embodiments, the disinfection treatment is conducted using UV light disinfection. By virtue of UV light disinfection, the efficiency of the disinfection treatment is improved without undesired effects on the vanilla pod.

In step (b) of this embodiment (which is referred to as “the sealing step 11” hereinafter), the container may be a vacuum bag 20. In certain embodiments, the disinfected vanilla pod is placed in a vacuum bag 20, followed by conducting air evacuation to generate a negative pressure of 75 kPa inside the vacuum bag 20, so as to achieve sealing. By virtue of the sealing step 11, the problem of excessive drying caused by evaporation of water and contamination of vanilla pods by mold and bacteria during processing can be avoided, thereby greatly reducing the chance of vanilla pods spoiling during processing.

In certain embodiments, in step (c) of this embodiment (which is referred to as the killing step 12″ hereinafter), the killing treatment may be conducted by subjecting the sealed container containing the disinfected vanilla pod to a heating treatment in hot water at a temperature ranging from ° C. to 90° C. for a time period ranging from 20 seconds to 60 seconds. In an exemplary embodiment, the container used in the sealing step 11 is a vacuum n bag 20, and in the killing step 12, the sealed vacuum bag 20 containing the disinfected vanilla pod is subjected to a heating treatment in hot water at a temperature of 80° C. for 30 seconds. It should be noted that if the heating treatment is conducted at a temperature of greater than 90° C., the released enzymes will be denatured, thereby reducing the efficiency of subsequent processing steps.

In certain embodiments, during the killing step 12, the killing treatment may be conducted by subjecting the sealed container containing the disinfected vanilla pod to a freezing treatment at a temperature ranging from −4° C. to −80° C. for a time period ranging from 1 hour to 3 hours.

in certain embodiments, in step (d) of this embodiment (which is referred to as “the synthesis step 13” hereinafter), the heating treatment is conducted by placing the sealed vacuum bag 20 containing the killed vanilla pod in an oven 30 for several months. Since the killed vanilla pod sealed in the vacuum bag 20, temperature of the oven will not cause moisture to be evaporated, thereby avoiding excessive drying of the killed vanilla pod and affecting the synthesis of aroma molecules.

In an exemplary embodiment, the killed vanilla pod in the sealed vacuum bag 20 is subjected to a heating treatment in an oven 30 at a temperature of 60° C., so that the enzymes can efficiently react with components in the killed vanilla pod to synthesize aroma molecules under stable conditions, without the need for introducing oxygen or other additional reactants.

The processed vanilla pods obtained by the first embodiment of the method according to the Present disclosure are confirmed to have a vanillin content of 2.71 g/100 g, indicating that the aroma molecules of the processed vanilla pods meet the specification requirements (i.e., the aroma of the processed vanilla pods meets the quality and level required on the market). On the contrary, the processed vanilla pods obtained using other methods (i.e., not using the first embodiment of the method according to the present disclosure) have the problem of premature drying and have a vanillin content of only 0.34 g/100 g.

In addition, by virtue of the sealing step 11, the disinfected vanilla pod is sealed in the vacuum bag 20 during the processing of the method of the present disclosure, thereby greatly reducing spoilage and mildew caused by improper moisture control, exposure to outside air, bacteria, and mold.

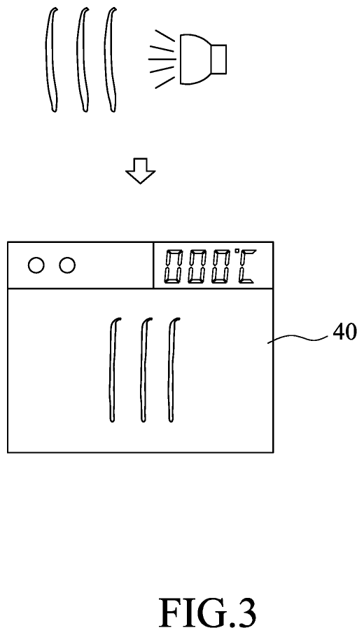

Referring to FIGS. 1 and 3, in the sealing step 11 of another variation of this embodiment, the container may be an airtight oven 40 whose temperature can be adjusted. In certain embodiments, the disinfected vanilla pod is placed in an airtight oven 40, followed by adjusting the temperature of the airtight oven 40 to 75° C. for 10 minutes, so as to conduct the killing step 12. Next, the temperature of the airtight oven 40 is adjusted to 60° C., so that the killed vanilla pod can be directly subjected to the synthesis step 13 in the airtight oven 40 without taking them out.

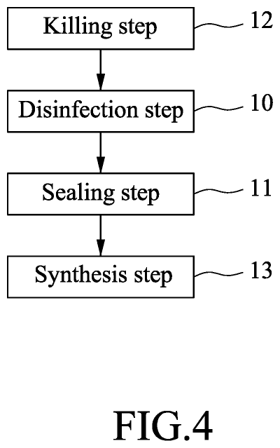

In addition, referring to FIG. 4, a second embodiment of the method according to the present disclosure includes:
(a) subjecting the vanilla pod to a killing treatment, so as to destroy the cellular tissues of the vanilla pod and release enzymes from the cellular tissues;
(b) subjecting the killed vanilla pod to a disinfection treatment to eliminate bacteria and mold on the surface of the killed vanilla pod
(c) placing the disinfected vanilla pod obtained in step (b) in a container, followed by sealing to keep away from air; and
(d) subjecting the disinfected vanilla pod in the sealed container to a heating treatment at a temperature ranging from 40° C. to 60° C., so that the enzymes react with the components in the disinfected vanilla pod to synthesize aroma molecules.

The second embodiment differs from the first embodiment in that, the killing step 12 is performed first, and then the disinfection step 10 and the sealing step 11 are performed in sequence. In the killing step 12 of this embodiment, if the hot water treatment is used, the effect of preventing the vanilla pod from contacting the air can be achieved, and if the freezing treatment is used, the growth of microorganisms can be avoided. Therefore, even if the vanilla pod is not sealed in the killing step 12, the subsequent synthesis step 13 would not be adversely affected, and effect similar to that of the first embodiment can be achieved. In particular, the second embodiment can be performed when a thermal-insulating vacuum bottle is used as an airtight container. The killing step 12 is performed before the vanilla pod is placed in the airtight container to ensure the release of the enzymes.

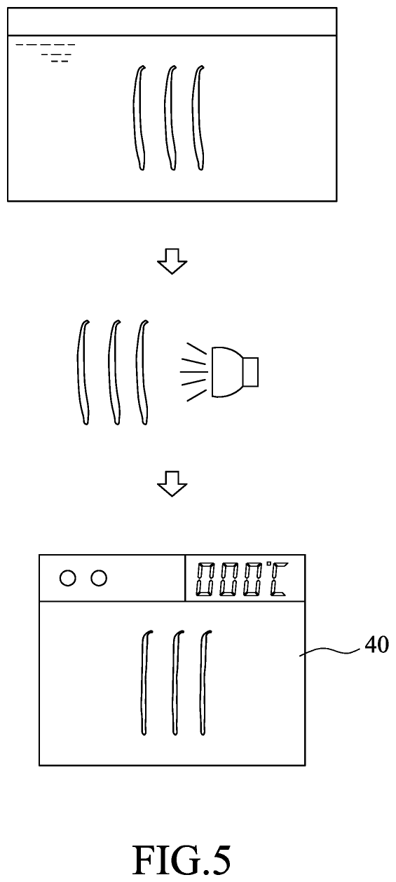

Referring to FIGS. 4 and 5, in the sealing step 11 of another variation of this embodiment, the container may be an airtight oven 40 whose temperature can be adjusted. The airtight oven 40 can achieve the purpose of isolating external interference factors, and the temperature required for the synthesis step 13 can be provided in the airtight oven 40, thereby directly performing the synthesis step 13, The second embodiment not only allows the vanilla pod to produce excellent aroma molecules, but also allows the sealing step 11 and the synthesis step 13 to be performed in an efficient manner.

Summarizing the above results, it is clear that by virtue of the sealing step 11, the vanilla pod is subjected to the synthesis step 13 under conditions of isolation from outside air, moisture, and microorganisms, thereby greatly reducing the problems of spoilage and premature drying. In addition, the method for processing a vanilla pod of the present disclosure can provide a stable synthesis environment with low external interference factors, and thus has the advantages of high yield, and synthesis of aroma molecules having excellent and consistent quality.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for processing a vanilla pod, comprising:
(a) placing the vanilla pod in a container, followed by sealing to keep away from air;
(b) subjecting the vanilla pod in the sealed container to a killing treatment, so as to destroy cellular tissues of the vanilla pod and release enzymes from the cellular tissues; and
(c) subjecting the killed vanilla pod in the sealed container to a heating treatment at a temperature ranging from 40° C. to 60° C., so that the enzymes react with components in the killed vanilla pod to synthesize aroma molecules.

2. The method according to claim 1, wherein before performing step (a), the vanilla pod is subjected to a disinfection treatment so as to eliminate bacteria and mold on a surface of the vanilla pod.

3. The method according to claim 2, wherein the disinfection treatment is conducted using a disinfection method selected from the group consisting of ultraviolet (UV) light disinfection, alcohol disinfection, hypochlorous acid disinfection, and combinations thereof.

4. The method according to claim 1, wherein in step (a), the vanilla pod is placed in a vacuum bag, followed by conducting air evacuation to generate a negative pressure ranging from 70 kPa to 80 kPa inside the vacuum bag, so as to achieve sealing.

5. The method according to claim 1, Wherein in step (b), the killing treatment is conducted by subjecting the sealed container containing the vanilla pod to a heating treatment in hot water at a temperature ranging from 70° C. to 90° C. for a time period ranging from 20 seconds to 60 seconds.

6. The method according to claim 1, wherein in step (b), the killing treatment is conducted by subjecting the sealed container containing the vanilla pod to a freezing treatment at a temperature ranging from −4° C. to −80° C. for a time period ranging from 1 hour to 3 hours.

7. The method according to claim 1, wherein in step (c), the heating treatment conducted by placing the sealed container containing the killed vanilla pod in an oven.

8. The method according to claim 1, wherein in step (a), the container is an airtight oven, and steps (b) and (c) are performed in the airtight oven in sequence.

9. The method according to claim 8, wherein in step (b), the killing treatment is conducted at a temperature ranging from 70° C. to 80° C. for a time period ranging from 5 minutes to 20 minutes.

10. A method for processing a vanilla pod, comprising:
(a) subjecting the vanilla pod to a killing treatment, so as to destroy cellular tissues of the vanilla pod and release enzymes from the cellular tissues;
(b) placing the killed vanilla pod obtained in step (a) in a container, followed by sealing to keep away from air; and
(c) subjecting the killed vanilla pod in the sealed container to a heating treatment at a temperature ranging from 40° C. to 60° C., so that enzymes react with components in the killed vanilla pod to synthesize aroma molecules.

11. The method according to claim 10, wherein before performing step (b), the killed vanilla pod is subjected to a disinfection treatment to eliminate bacteria and mold on a surface of the killed vanilla pod.

12. The method according to claim 11, wherein the disinfection treatment is conducted using a disinfection method selected from the group consisting of ultraviolet (UV) light disinfection, alcohol disinfection, hypochlorous acid disinfection, and combinations thereof.

13. The method according to claim 10, wherein in step (b), the killed vanilla pod is placed in a vacuum bag, followed by conducting air evacuation to generate a negative pressure ranging from 70 kPa to 80 kPa inside the vacuum bag, so as to achieve sealing.

14. The method according to claim 10, wherein in step (a), the killing treatment is conducted by subjecting the vanilla pod to a heating treatment in hot water at a temperature ranging from 70° C. to 90° C. for a time period ranging from 20 seconds to 60 seconds.

15. The method according to claim 10, wherein in step (a), the killing treatment is conducted by subjecting the vanilla pod to a freezing treatment at a temperature ranging from −4° C. to −80° C. for a time period ranging from 1 hour to 3 hours.

16. The method according to claim 10, wherein in step (c), the heating treatment is conducted by placing the sealed container containing the killed vanilla pod in an oven.

17. The method according to claim 10, wherein in step (b), the container is an airtight oven, and after performing step (b), step (c) is directly performed in the airtight oven.
